(19) United States
(12) Patent Application Publication
(10) Pub. No.: US 2007/0106379 A1
(43) Pub. Date: May 10, 2007

(54) INTRAOCULAR LENS

(75) Inventor: Arthur Messner, Schnaittach (DE)

Correspondence Address: BARNES & THORNBURG LLP, P.O. BOX 2786, CHICAGO, IL, 60690-2786, US

(73) Assignee: DR. SCHMIDT INTRAOKULARLINSEN GMBH, St. Augustin (DE)

(21) Appl. No.: 11/555,592

(22) Filed: Nov. 1, 2006

(30) Foreign Application Priority Data
Nov. 4, 2005 (DE) 102005053078

(51) Int. Cl. A61F 2/16

(52) U.S. Cl. 623/6.17; 623/6.51

(57) ABSTRACT

In an intraocular lens for implantation in the anterior chamber of an eye, to improve the compatibility and the imaging characteristics, a peripheral surface extending between an anterior optic surface and a posterior optic surface extends parallel to an optical axis and optic edges defining the peripheral surface are of a sharp-edged configuration. A design of this type of the intraocular lens reduces imaging defects.

### BACKGROUND OF THE INVENTION

1. Field of the Invention

The invention relates to an intraocular lens for implantation in the anterior chamber of an eye, in particular a human eye.

2. Background Art

Intraocular lenses for implantation in the anterior chamber of an eye have been known for a long time and are implanted in the human eye either after removing the natural lens as a replacement for said lens or in addition to the natural lens to correct defective vision. Intraocular lenses which are additionally implanted without removing the natural lens are also termed phaco intraocular lenses. In the development and application of intraocular lenses of this type, there is a constant need to improve the compatibility and the imaging characteristics of the implanted intraocular lenses.

### SUMMARY OF THE INVENTION

The object of the invention is to provide an intraocular lens for implantation in the anterior chamber of an eye, which lens avoids post-operative complications and imaging defects in the eye.

This object is achieved by an intraocular lens for implantation in an eye, with an optic which consists of an optic material and has an optical axis, an anterior optic surface extending continuously and transversely to the optical axis, a posterior optic surface extending continuously and opposite the anterior optic surface, a peripheral surface extending between the optic surfaces and along the optical axis, a sharp-edged anterior optic edge defining the peripheral surface and the anterior optic surface, and a sharp-edged posterior optic edge defining the peripheral surface and the posterior optic surface, and with at least two haptics which are attached to the optic, consist of a haptic material and have in each case two haptic arms for attaching the intraocular lens to the iris. The core of the invention consists in the fact that the anterior and posterior optic edges are configured sharply as a transition between the optic surfaces and the peripheral surface and the peripheral surface extends substantially along the optical axis. This measure basically prevents imaging defects caused by the optic edges and the peripheral surface. As a result of the sharp-edged transition to the peripheral surface, the optic surfaces may be used over the entire surface, i.e. also in the peripheral region thereof. The optic may be attached to the iris of the eye in a simple and very compatible manner by the haptic arms.

Additional features and details of the invention are provided in the description of an embodiment with reference to the drawings.

### BRIEF DESCRIPTION OF THE DRAWINGS

FIG. 1 an anterior view of an intraocular lens,

FIG. 2 a side view of the intraocular lens of FIG. 1,

FIG. 3 a cross-section through the intraocular lens along the section line III-III in FIG. 1,

FIG. 4 an enlarged detail of the intraocular lens of FIG. 3, and

FIG. 5 a cross-section through the intraocular lens along the section line V-V in FIG. 1 with a simplified illustration of the haptic arms.

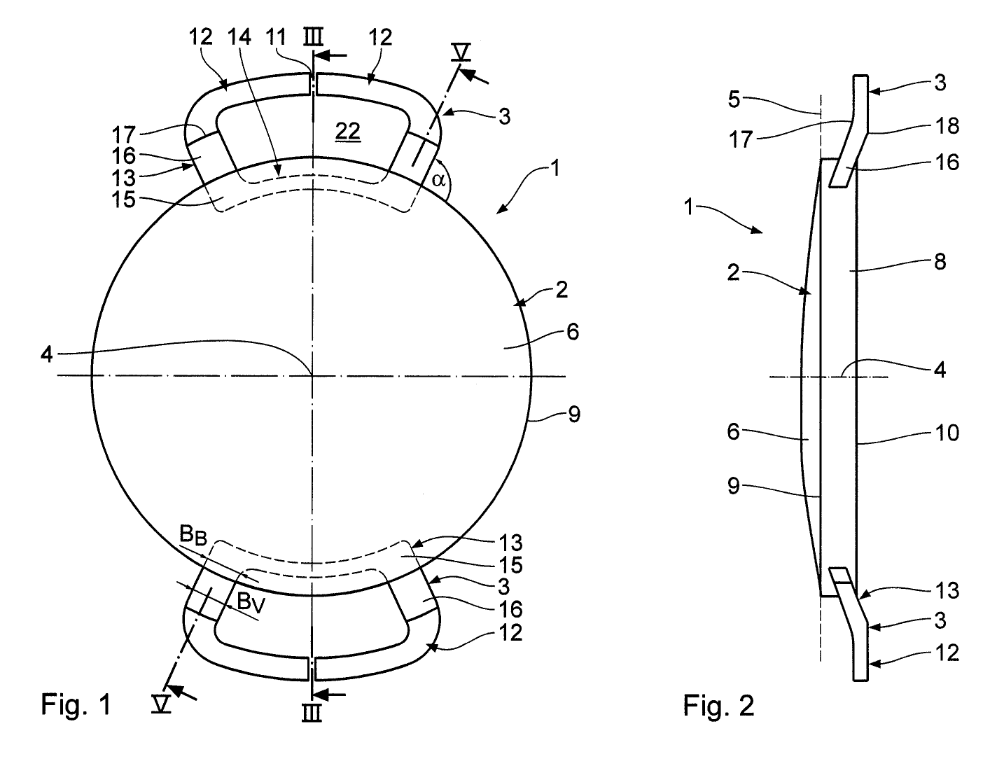

### DESCRIPTION OF THE PREFERRED EMBODIMENT

An intraocular lens 1 is composed of several parts and has a central optic 2 and haptics 3 attached thereto. The optic 2 has in the centre an optical axis 4 and a lens plane 5 extending through the optic 2 perpendicularly on the optical axis 4.

The optic 2 comprises an anterior optic surface 6 facing, when implanted, the cornea of the eye and a posterior optic surface 7 facing the iris of the eye and opposing the anterior optic surface 6 relative to the lens plane 5. The optic surfaces 6, 7 extend substantially transversely to the optical axis 4 and are constant, i.e. the optic surfaces 6, 7 do not have any Fresnel zones because of their constant path. The surfaces 6, 7 preferably run in a continuously curved manner. An annular peripheral surface 8 runs concentrically and parallel to the optical axis 4 and extends between the optic surfaces 6, 7. The peripheral edge 8 is delimited by a circular anterior optic edge 9 in the direction of the anterior optic surface 6 and by a circular posterior optic edge 10 in the direction of the posterior optic surface 7.

The anterior optic edge 9 is of a sharp configuration and extends along a partial arc of a circle with a radius R₁ of less than 100 μm, particularly less than 50 μm and in particular less than 10 μm. The posterior optic edge 10 is also of a sharp configuration and extends along a partial arc of a circle with a radius R₂ of less than 100 μm, particularly less than 50 μm and in particular less than 10 μm.

The anterior optic surface 6 is curved in convex manner relative to the lens plane 5 and has a radius of curvature R_{A} ranging from 3 mm to 90 mm, particularly from 4 mm to 80 mm and in particular from 5 mm to 70 mm. Alternatively, the anterior optic surface 6 may also be curved in concave manner relative to the lens plane 5 and may have a radius of curvature R_{A} within the aforementioned ranges. The posterior optic surface 7 is curved in concave manner relative to the lens plane 5 and has a radius of curvature R_{P} ranging from 4 mm to 20 mm, particularly from 6 mm to 16 mm and in particular from 8 mm to 12 mm.

The optic surfaces 6, 7 are configured in such a way that it is possible to describe in mathematical terms a wavefront deformation substantially with Zernike polynomials up to an order of 8, particularly up to an order of 6, and in particular up to an order of 4. Zernike polynomials are a qualitative and quantitative measure of the imaging defects of the optic 2.

The optic 2 is produced in one piece from an optic material. Silicone, for example, which has a refractive index of approximately 1.43 is used as the optic material. Alternatively, a hydrophilic acrylate having a refractive index of 1.46 or a high-refractive optic material having a refractive index of greater than 1.5 may be used as the optic material.

The two haptics 3 attached to the optic 2 extend radially outwards and are positioned opposite one another relative to the optical axis 4. Alternatively, it is also possible to provide a different number of haptics provided that they are distributed uniformly over the circumference of the optic. The haptics 3 are identical and are attached to the optic 2, so that in the following only one haptic 3 will be described in detail.

The haptic 3, formed in one piece, has two haptic arms 12 defining a nipping gap 11 and a haptic strap 13 connecting the haptic arms 12. The haptic strap 13 comprises an attachment portion 15 accommodated in an optic recess 14 and completely surrounded by the optic material, and exposed connecting portions 16 positioned on attachment portion 15. Said attachment portion 15 extends in the middle concentrically to the optical axis 4 and is angled off at the ends in such a way that it extends adjacently to the connecting portions 16 and extends radially outwards corresponding to said connecting portions 16. The haptic strap 13 penetrates the peripheral surface 8, considered circumferentially, at an angle α of approximately 90°. The haptic strap 13 extends substantially tangentially to and flush with the posterior optic surface 7 and extends up to an anterior haptic edge 17 and a posterior haptic edge 18. Seen along the optical axis 5, the connecting portions 16 enclose with the peripheral surface 8 an angle β adapted to the radius of curvature R_{P} of the posterior optic surface 7. The anterior haptic edge 17 and the posterior haptic edge 18 extend substantially parallel to the peripheral surface 8, the anterior haptic edge 17 being at a greater distance from the optical axis 4 than the posterior haptic edge 18 due to the inclined position of the haptic strap 13 relative to the lens plane 5.

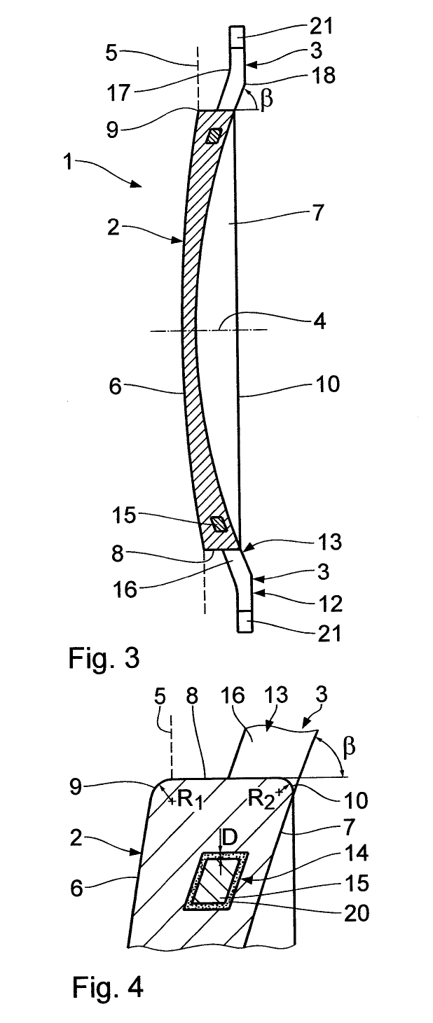

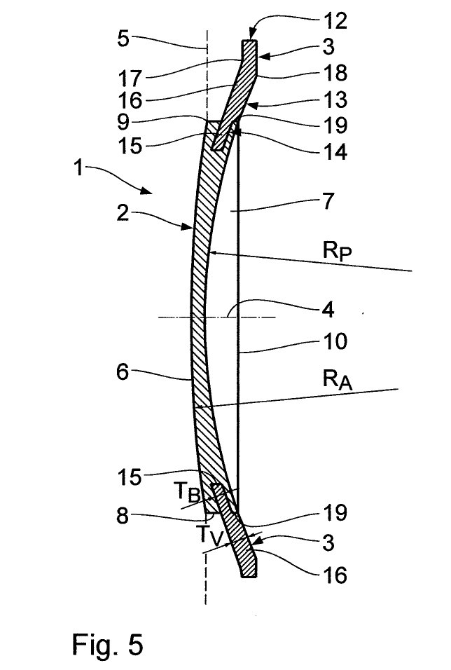

In the centre, the attachment portion 15 has a cross-section in the shape of a parallelogram, an upper and a lower side wall extending parallel to the peripheral surface 8, as shown in FIGS. 3 and 4. In the region of the peripheral surface 8, the cross-section of the attachment portion 15 increases in such a way that along the peripheral edge 8 a width B_{B} of the attachment portion 15 corresponds to a width B_{V} of the connecting portions 16. Seen along the optical axis 4, the connecting portions 16 have a depth T_{V} which is greater than a depth T_{B} of the attachment portion 15, so that the connecting portions 16 form a projection 19 which extends in the direction of the posterior optic edge 10 and rests against the peripheral surface 8, as shown in FIG. 5.

A coating 20 which is impermeable to light of the visible spectrum is positioned between the attachment portion 15 of the haptic strap 13 and the optic material. Said coating 20 has a thickness D of at least 0.5 μm, particularly at least 1 μm and in particular at least 5 μm. As an alternative to the coating, the surface of the attachment portion 15 may be roughened

Emanating from the anterior haptic edges 17 and the posterior haptic edges 18, the haptic arms 12 extend parallel to the lens plane 5. The haptic arms 12 extend, emanating from the haptic edges 17, 18, initially radially outwards and then bend in such a way that they extend substantially parallel to the peripheral surface 8 and towards one another. At their free ends, the haptic arms 12 have end faces 21 which extend perpendicularly to the lens plane 5 and laterally define the nipping gap 11.

To attach the intraocular lens 1 to the iris, the haptics 3 have on the end faces 21 an average surface roughness of at least 5 μm, particularly at least 10 μm and in particular at least 20 μm. The average surface roughness of the other haptic surfaces is less than 4 μm, particularly less than 3 μm and in particular less than 2.5 μm.

The peripheral surface 8 of the optic 2, the connecting portions 16 of the haptic strap 13 and the haptic arms 12 substantially define a haptic recess 22. This haptic recess 22 is connected to the nipping gap 11.

The haptics 3 are formed in one piece from a haptic material, PMMA in particular being used as haptic material. The haptic material has a first modulus of elasticity E_{H} and the optic material a second modulus of elasticity E_{O}, the ratio of the modules of elasticity E_{H}/E_{O} preferably being greater than 1.5, particularly greater than 2, and in particular greater than 3. The haptic material also has a first refractive index B_{H} and the optic material has a second refractive index B_{O}, the difference between the refractive indices B_{H}-B_{O} amounting to at least 0.03, particularly 0.06 and in particular 0.09.

The intraocular lens 1 is implanted in the anterior chamber of an eye. It is used either as a replacement for the natural lens which was removed, for example due to a cataract, or is used in addition to the natural lens in order to correct defective vision. The intraocular lens I is inserted into the anterior chamber of the eye through an incision in the cornea and is attached to the iris by the haptic arms 12. For this purpose, the iris is clamped between the haptic arms 12 in the nipping gap 11 in the haptics 3. The width of the nipping gap 11 and the average surface roughness of the end faces 21 are in such a way that the intraocular lens 1 is reliably attached to the iris by the haptics 3. The haptic arms 12 rest on the iris with their posterior lateral surfaces facing the iris so that the optic 2 is raised from the iris due to the haptic straps 13 extending obliquely to the lens plane 5, and is exposed. Since only the haptic arms 12 are in contact with the iris, the intraocular lens 1 has a good compatibility. The sharp-edged configuration of the optic edges 9, 10 and the peripheral surface 8 extending parallel to the optical axis 4 reduce imaging defects, so that the intraocular lens 1 has very good imaging characteristics. When forces are exerted on the haptics 3, the geometry of the haptics 3 and their attachment to the optic 2 allow a uniform introduction of such force into the optic 2, thereby reducing imaging defects due to a deformation of the optic 2. In addition, the coating 20 between the haptic material and the optic material reduces imaging defects caused by disturbing light effects, by virtue of the haptic straps 13 extending in the optic material.

What is claimed is:

1. Intraocular lens for implantation in an eye, with
a. an optic (2) which consists of an optic material and has
i. an optical axis (4),
ii. an anterior optic surface (6) extending continuously and transversely to the optical axis (4),
iii. a posterior optic surface (7) extending continuously and opposite the anterior optic surface (6),
iv. a peripheral surface (8) extending between the optic surfaces (6, 7) and along the optical axis (4),
v. a sharp-edged anterior optic edge (9) defining the peripheral surface (8) and the anterior optic surface (6), and
vi. a sharp-edged posterior optic edge (10) defining the peripheral surface (8) and the posterior optic surface (7), and
b. at least two haptics (3) which are attached to the optic (2), consist of a haptic material and have
i. in each case two haptic arms (12) for attaching the intraocular lens (1) to the iris.

2. Intraocular lens according to claim 1, wherein the anterior optic edge (9) extends along a partial arc of a circle with a radius (R₁) of less than 100 μm.

3. Intraocular lens according to claim 1, wherein the posterior optic edge (10) extends along a partial arc of a circle with a radius (R₂) of less than 100 μm.

4. Intraocular lens according to claim 1, wherein the optic surfaces (6, 7) are configured in such a way that it is possible to describe a wavefront deformation, caused by the intraocular lens, substantially with Zernike polynomials up to an order of 8.

5. Intraocular lens according to claim 1, wherein the haptic material has a first modulus of elasticity (E_{H}) and the optic material has a second modulus of elasticity (E_{O}), the ratio of the modules of elasticity (E_{H}/E_{O}) being greater than 1.5.

6. Intraocular lens according to claim 1, wherein a haptic strap (13) at least partly enclosed by the optic material is provided in each case to attach the at least two haptic arms (12) to the optic (2).

7. Intraocular lens according to claim 6, wherein a coating (20) impermeable to light of the visible spectrum is provided between the haptic strap (13) and the optic material.

8. Intraocular lens according to claim 7, wherein the coating (20) has a thickness (D) of at least 0.5 μm.

9. Intraocular lens according to claim 1, wherein the anterior optic surface (6) is curved in convex or concave manner relative to a lens plane (5) and has a radius of curvature (R_{A}) ranging from 3 mm to 90 mm.

10. Intraocular lens according to claim 1, wherein the posterior optic surface (7) is curved in concave manner relative to a lens plane (5) and has a radius of curvature (R_{P}) ranging from 4 mm to 20 mm.
